(19) United States
(12) Patent Application Publication
(10) Pub. No.: US 2007/0282281 A1
(43) Pub. Date: Dec. 6, 2007

(54) Piercing tip

(76) Inventors: J. Warner Ide, Hudson, WI (US); Jeffrey A. Crane, Warrenwood (AU)

Correspondence Address: Brooks & Cameron, PLLC, Suite 500, 1221 Nicollet Avenue, Minneapolis, MN, 55403, US

(21) Appl. No.: 11/804,792

(22) Filed: May 21, 2007

Related U.S. Application Data
(60) Provisional application No. 60/802,190, filed on May 19, 2006.

(51) Int. Cl. A61M 5/32

(52) U.S. Cl. 604/272

(57) ABSTRACT

The present disclosure includes various embodiments for a piercing tip and piercing tip systems. In one such embodiment, a piercing tip including a body portion, a barbed portion at a first end of the body portion, and a cap connected to the body portion.

### BACKGROUND

The treatment of slaughtered animals by injecting a treatment solution into the circulation system of the slaughtered animals can be used to provide for improved meat quality and improved yield, among other benefits. Such processing, also called “rinsing,” involves the removal of blood from the animal and the introduction of the treatment solution into the circulatory system.

An exemplary apparatus for administering a treatment solution to animals such as cattle, horses, hogs, poultry, deer, buffalo, sheep among others is described in U.S. Pat. No. 5,007,336, and is incorporated herein by reference. An exemplary slaughtering process is described in U.S. Pat. No. 5,964,656, and is incorporated herein by reference.

Such treatment processes can be complicated, for example, by the desirability of maintaining sanitation of the devices put into contact with the animals. Another complication involves retaining an injection device in the animal's circulatory system during treatment. Further, in some instances it may be difficult to provide the treatment in timely way, since some insertion locations that are used provide indirect access to an intended location of the device.

### BRIEF DESCRIPTION OF THE DRAWINGS

FIG. 1 illustrates a piercing tip according to an embodiment of the present disclosure.

### DETAILED DESCRIPTION

In the following detailed description of the present disclosure, reference is made to the accompanying drawings that form a part hereof, and in which is shown by way of illustration how various embodiments of the disclosure may be practiced. These embodiments are described in sufficient detail to enable those of ordinary skill in the art to practice the embodiments of this disclosure, and it is to be understood that other embodiments may be utilized and that process, electrical, or mechanical changes may be made without departing from the scope of the present disclosure.

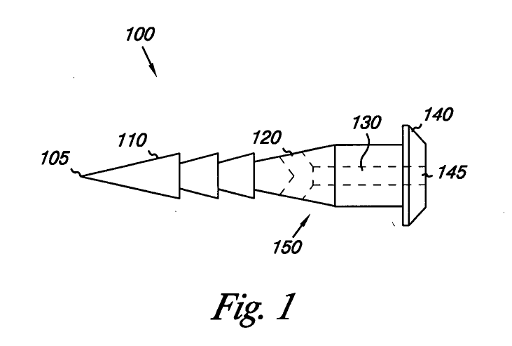

FIG. 1 illustrates a piercing tip according to an embodiment of the present disclosure. In the embodiment shown in FIG. 1, the piercing tip 100 includes a barbed portion 110, a body portion 150, and a cap 140.

As shown in the embodiment of FIG. 1, the barbed portion 110 includes a number of barbs and terminates in a piercing point 105. The one or more barbs can be shaped in any of various barb shapes that are suitable for maintaining the tip in place at the insertion location (e.g., with a portion of the body 150 in fluid communication with a portion of the circulatory system, such as the heart, an artery, a vein, etc.).

In various embodiments, the piercing point can be used to pierce through the body of the animal and into a portion of the circulatory system, such as into the heart of a slaughtered animal (e.g., a cow, a pig, a sheep, or a turkey, among various other animals). In embodiments utilizing barbs, the barbs can be used to retain the piercing tip in its insertion location, such as in the heart, during introduction of a rinse solution. In some embodiments, the piercing point can be used to pierce various other circulatory locations including the femoral artery, among other circulatory system locations.

The piercing tip can be made of various materials including various metals and/or plastics, among other materials. Some materials may be beneficial in increasing the durability of the tip, increasing the piercing ability of the tip, providing better sanitization of the tip, and/or allowing the tip to be more readily reusable or disposable, among other benefits.

In various embodiments, the rinse solution can be a chilled solution that can cause the circulatory tissue through which the tip is pierced, such as the heart wall, to contract around the barbs of tip 100 during introduction of the solution into the heart. In such situations, the barbs may be smaller or less pronounced than in the embodiment illustrated in FIG. 1, since the contraction of the tissue may aid in holding the tip in place.

In various embodiments, such as that shown in FIG. 1, the piercing tip can include a lumen 130 through body 150 for delivery of the rinse solution. In the embodiment illustrated in FIG. 1, the lumen 130 is bifurcated near barbed portion 110 such that fluid can be delivered into the circulatory system (e.g., the heart) through side ports 120. The side ports can be positioned at various angles (e.g., they can be directed toward the barbed portion 110 as shown in FIG. 1 or can be directed toward the cap 140, among various other directions).

Embodiments are not limited to having multiple side ports, nor are the embodiments limited to a bifurcated lumen. For instance, the body could include a single port (e.g., through a side of the body or the tip) or more than two ports, among other configurations.

In some embodiments, as discussed above, the lumen 130 can pass through at least a portion of the tip portion 110. In such embodiments, the lumen may or may not be bifurcated and the tip may or may not include side ports.

In the embodiment shown in FIG. 1, the barbed tip 110 includes a tiered barb structure having multiple barbs that can facilitate retention of the piercing tip in the circulatory system of an animal during introduction of a rinse solution. In various embodiments, and as shown in the embodiment of FIG. 1, the tiered barb structure can be arranged in a linear manner. While the embodiment of FIG. 1 shows tip 110 as including three tiered barbs, various embodiments can have more or fewer than three barbs.

In the embodiment illustrated in FIG. 1, the piercing tip includes a connection 145 at an end of body 150 opposite tip 110. The connection 145 on the end face can be used for attaching to a cannula, for example, to provide fluid communication with a reservoir of rinse solution.

The type of connection can be any of various suitable types of connections. For example, in various embodiments, the connection can be a threaded connection or other type of mechanical connection. In some embodiments, the connection can be a frictional connection, among other suitable connection types.

As illustrated in the embodiment illustrated in FIG. 1, the piercing tip can include a cap 140 at an end of body 150 opposite tip 110. In various embodiments, the cap can be used to limit the distance that the piercing tip can be inserted into the animal, among other uses. For instance, in various embodiments, such as that shown in FIG. 1, the cap can extend outwardly from the piercing tip body and can interact with the outer heart wall to prevent the cap from entering the heart.

In some embodiments, the piercing tip 100 can be disposable. In such embodiments, the piercing tip can remain in the body of the animal after the animal has been rinsed. For example, in some embodiments, the cannula and tip attachment can be designed such that pulling or twisting a cannula body attached to the tip with a sufficient force can be used to detach the cannula from the tip at connection 145.

The dimensions of the tip can be any suitable dimensions. For example, in various embodiments, the barbed portion 110 can be about 45 mm long, the body 150 can be about 40 mm long, the lumen 130 and/or side ports 120 can be about 5 mm in diameter, and the cap 140 can be about 26 mm in diameter. In such an embodiment, the piercing tip 100 can have a total length of about 90 mm. However, embodiments are not limited to these exemplary dimensions.

In the foregoing Detailed Description, various features are grouped together in a single embodiment for the purpose of streamlining the disclosure. This method of disclosure is not to be interpreted as reflecting an intention that the disclosed embodiments of the present disclosure have to use more features than are expressly recited in each claim.

Rather, as the following claims reflect, inventive subject matter lies in less than all features of a single disclosed embodiment. Thus, the following claims are hereby incorporated into the Detailed Description, with each claim standing on its own as a separate embodiment.

What is claimed:

1. A piercing tip, comprising:
a body portion;
a barbed portion at a first end of the body portion; and
a cap connected to the body portion.

2. The piercing tip of claim 1, where the barbed portion includes a number of barbs and terminates in a piercing point.

3. The piercing tip of claim 1, where at least one of the number of barbs is shaped to maintain the tip in place at an insertion location.

4. The piercing tip of claim 1, where at least one of the number of barbs is shaped to maintain the tip in place at an insertion location, where at least some of the body portion is in fluid communication with a portion of a circulatory system of an animal.

5. The piercing tip of claim 1, where at least one of the number of barbs is shaped to maintain the tip in place at an insertion location, where at least some of the body portion is in fluid communication with a portion of a circulatory system selected from a group of locations including a heart, an artery, and a vein.

6. The piercing tip of claim 1, where at least one of the number of barbs is shaped to retain the piercing tip in an insertion location during introduction of a rinse solution through a lumen in the piercing tip.

7. The piercing tip of claim 1, where barbed portion includes a tiered barb structure having multiple barbs.

8. The piercing tip of claim 7, where the number of barbs on the tiered barb structure are arranged in a linear manner.

9. The piercing tip of claim 1, where the piercing tip includes a connection at an end of the body opposite a tip portion of the body.

10. The piercing tip of claim 9, where the connection is shaped for attaching to a cannula to provide fluid communication with a reservoir of rinse solution.

11. The piercing tip of claim 10, where the connection is shaped such that a pulling or twisting force can be used to detach the cannula from the tip portion at the connection.

12. The piercing tip of claim 1, where the cap is shaped to limit the distance that the piercing tip can be inserted into an animal.

13. The piercing tip of claim 1, where the cap is shaped to extend outwardly from the piercing tip body and can interact with an outer heart wall to prevent the cap from entering a heart.

14. A piercing tip, comprising:
a body portion having a piercing point;
a lumen formed through the body; and
a cap.

15. The piercing tip of claim 14, where the piercing point is shaped to pierce through the body of an animal and into a portion of the circulatory system.

16. The piercing tip of claim 14, where the piercing point is shaped to pierce through the body of the animal and into a heart of a slaughtered animal.

17. The piercing tip of claim 14, where the piercing point is shaped to pierce through the body of an animal and into a femoral artery.

18. The piercing tip of claim 14, where the piercing tip is made from a metal material.

19. The piercing tip of claim 14, where the piercing tip is made from a plastic material.

20. A piercing tip system, comprising:
a piercing tip having;
a body portion having a piercing point;
a lumen formed through the body; and
a cap; and
a solution injection mechanism for injecting a solution into the lumen formed through the body.

21. The piercing tip system of claim 20, where the solution is a chilled solution that can cause circulatory tissue through which the tip is pierced to contract around the body portion during introduction of the solution into a circulatory system.

22. The piercing tip system of claim 20, where the lumen is bifurcated.

23. The piercing tip system of claim 20, where the body includes a number of side ports in fluid communication with the lumen.

24. The piercing tip system of claim 23, where the side ports are oriented to direct solution from the lumen toward the piercing point.

25. The piercing tip system of claim 20, where a portion of the lumen is formed through at least part of a tip portion of the body.
